(19) United States
(12) Patent Application Publication
(10) Pub. No.: US 2025/0060358 A1
(43) Pub. Date: Feb. 20, 2025

(54) DIAGNOSIS AND TREATMENT OF HISTOPLASMOSIS

(71) Applicant: The Brigham and Women’s Hospital, Inc., Boston, MA (US)

(72) Inventor: Sophia Koo, Brookline, MA (US)

(21) Appl. No.: 18/692,269

(22) PCT Filed: Sep. 14, 2022
(86) PCT No.: PCT/US2022/076390

§ 371 Date: Mar. 14, 2024

Related U.S. Application Data
(60) Provisional application No. 63/243,950, filed on Sep. 14, 2021.

(51) Int. Cl. G01N 33/497; A61K 31/496; A61K 31/7048; C12Q 1/18; G01N 30/72; G01N 30/02

(52) U.S. Cl. CPC G01N 33/4977; A61K 31/496; A61K 31/7048; C12Q 1/18; G01N 30/7206; G01N 2030/025; G01N 2333/37; G01N 2800/26; G01N 2800/52

(57) ABSTRACT

Methods for diagnosing, treating, and monitoring the treatment of histoplasmosis. The methods can include detecting the presence of one or more volatile organic compounds (VOCs) in the breath of subjects suspected of having histoplasmosis.

### CLAIM OF PRIORITY

This application claims the benefit of U.S. Provisional Patent Application Ser. No. 63/243,950, filed on Sep. 14, 2021. The entire contents of the foregoing are hereby incorporated by reference.

### FEDERALLY SPONSORED RESEARCH OR DEVELOPMENT

This invention was made with Government support under Grant No. R21 AI133330 awarded by the National Institutes of Health. The Government has certain rights in the invention.

### TECHNICAL FIELD

Provided herein are methods for diagnosing, treating, and monitoring the treatment of histoplasmosis. The methods can include detecting the presence of one or more volatile organic compounds (VOCs) in the breath of subjects suspected of having histoplasmosis.

### BACKGROUND

Histoplasma capsulatum is a dimorphic fungal pathogen capable of causing acute pulmonary disease in otherwise healthy individuals and lethal disease in immunocompromised humans (Ampel, 1996, Emerg. Infect. Dis., 2: 109-116; Eissenberg, 1994, The Interplay Between Histoplasma Capsulatum and Its Host Cells, Vol, I, Ch. 6, W. B. Saunders Company, Ltd. London, UK; Wheat et al., 1985, Am. J. Med., 78: 203-210). In its most serious form, the infection disseminates throughout the body. Disseminated histoplasmosis, coinciding with laboratory evidence of HIV infection, is regarded sufficient for a diagnosis of AIDS (Castro et al., 1992, MMRW 41: 1-14). Although AIDS currently represents the most prevalent immunocompromising disease of humans, a variety of other conditions or medical treatments can impair the human immune system and create susceptibility to diseases caused by the primary pathogen H. capsulatum and associated opportunistic pathogens (Goodwin et al., 1981, Medicine (Baltimore) 60: 231-266). These predisposing conditions include advanced age, diabetes, cancer chemotherapy, or immunosuppression induced to prevent rejection of transplanted organs (Wheat et al., 1982, Ann. Intern. Med., 96: 159-163; Davies et al., 1978, Am. J. Med. 64: 94-100).

### SUMMARY

As described herein, the present inventors have (1) identified a unique, profile of volatile organic compounds (VOCs) produced by Histoplasma capsulatum, (2) demonstrated that GC-MS can be used for the rapid discrimination between various fungal species using pattern-based detection of VOC profiles, and (3) accurately identified patients with histoplasmosis via direct detection of a pattern of Histoplasma capsulatum VOCs in their breath, including a combination of cyperene, 1R,4aR,8aR)-2, 5,5,8a-Tetramethyl-4,5,6,7,8,8a-hexahydro-1H-1,4a-methanonaphthalene, and viridiflorol.

Thus in a first aspect, the invention provides methods for diagnosing a subject with histoplasmosis, the method comprising: obtaining a sample comprising breath of a subject or suspected of comprising Histoplasma capsulatum isolated from a subject; detecting the presence in the sample of one, two, or all three volatile organic compounds (VOCs) produced by Histoplasma capsulatum in a sample comprising breath from the subject or headspace from a culture suspected of comprising Histoplasma capsulatum isolated from the subject, wherein the VOCs are selected from the group consisting of cyperene, 1R,4aR,8aR)-2,5,5,8a-Tetramethyl-4, 5,6,7,8,8a-hexahydro-1H-1,4a-methanonaphthalene, and viridiflorol; and diagnosing a subject as having histoplasmosis when there are one, two, or all three of the VOCs present in the sample.

In another aspect, the invention provides methods of treating a subject who has histoplasmosis, the method comprising: obtaining a sample comprising breath of a subject or headspace from a culture suspected of comprising Histoplasma capsulatum isolated from a subject; detecting the presence in the sample one, two, or all three VOCs selected from the group consisting of cyperene, 1R,4aR,8aR)-2,5,5,8a-Tetramethyl-4, 5,6,7,8,8a-hexahydro-1H-1,4a-methanonaphthalene, and viridiflorol; and administering an antifungal treatment to a subject who has one, two, or all three VOCs selected from the group consisting of cyperene, 1R,4aR,8aR)-2,5,5,8a-Tetramethyl-4, 5,6,7,8,8a-hexahydro-1H-1,4a-methanonaphthalene, and viridiflorol.

In some embodiments, of any of the methods described above the sample is a sample of a subject's breath.

Another aspect of the invention described herein are methods of monitoring efficacy of a treatment for histoplasmosis in a subject, the method comprising: determining a first level of one, two, or all three volatile organic compounds (VOCs) produced by Histoplasma capsulatum in a sample comprising breath from the subject or headspace from a culture suspected of comprising Histoplasma capsulatum isolated from the subject, wherein the VOCs are selected from the group consisting of cyperene, 1R,4aR,8aR)-2,5,5,8a-Tetramethyl-4,5,6,7,8,8a-hexahydro-1H-1,4a-methanonaphthalene, and viridiflorol, in the subject; administering a treatment for histoplasmosis to the subject; determining a second level of the VOCs in a sample obtained after administration of the treatment to the subject; and comparing the first and second levels of VOCs, wherein a decrease in the VOCs indicates that the treatment has been effective in treating the histoplasmosis in the subject, and an increase or no change indicates that the treatment has not been effective in treating the histoplasmosis in the subject.

In some embodiments, of any of the methods described above the treatment comprises administration of one or more doses of one or more antifungal compounds.

In another aspect, provided are methods of identifying a candidate compound for the treatment of histoplasmosis, the method comprising: providing a test culture comprising Histoplasma capsulatum; detecting a baseline level of fungal VOCs in the headspace of the culture in the absence of the test compound, wherein the VOCs are selected from the group consisting of cyperene, 1R,4aR,8aR)-2,5,5,8a-Tetramethyl-4, 5,6,7,8,8a-hexahydro-1H-1,4a-methanonaphthalene, and viridiflorol, in the subject; contacting the test culture with a test compound; determining a second level of the VOCs in a the test culture; comparing the second level of VOCs to the baseline level; and identifying a test compound that decreases levels of fungal VOCs in the test culture as a candidate compound for the treatment of histoplasmosis.

In some embodiments, of any of the methods described, wherein determining the presence of a VOC comprises assaying the sample to detect the presence the VOC. In some embodiments, wherein assaying the sample to detect the presence the VOC comprises using a gas chromatography-mass spectrometry (GC-MS) method. In some embodiments, wherein the spectrophotometry method is mobility spectrometry (IMS) or differential mobility spectrometry (DMS).

In some embodiments of the various methods described herein, the subject is a human.

In some embodiments of the various methods described herein, the antifungal compound is amphotericin B. In some embodiments, the antifungal compound is an azole antifungal compound. In some embodiments, the azole compound is itraconazole. In some embodiments, the antifungal compounds are amphotericin B and an azole antifungal compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a set of three graphs showing TDU GC-MS/MS spectral comparison in histoplasmosis vs. the other invasive mycoses aspergillosis or mucormycosis. The peaks are: A: Cyperene; B: (1R,4aR,8aR)-2,5,5,8a Tetramethyl-4, 5,6,7,8,8a-hexahydro-1H-1,4a-methanonaphthalene; C: viridiflorol; D: 1H-Indene, 2,3,3a,4-tetrahydro-3,3a,6-trimethyl-1-(1-methylethyl)-; E: 62 -funebrene; F: trans-α-bergamotene; G: eremophilene; H: spathulenol; I: cedrene; J: cedranoxide, 8,14-.

### DETAILED DESCRIPTION

Histoplasmosis, caused by the dimorphic fungus Histoplasma capsulatum, is a globally-distributed endemic infection with cases found in America, Asia, Africa, and Europe.¹ Central America, parts of South America and the US Midwest, particularly the Ohio and Mississippi river² valleys account for a large portion of cases in older adults (6.1 cases per 100000 person-years)³. It is especially prevalent in HIV patients and it can mimic or coexist with other endemic entities such as tuberculosis and other invasive opportunistic fungal diseases like aspergillosis or mucormycosis. This makes its diagnosis a bigger challenge.

The respiratory cultures are the gold standard but they are slow-growing (4-8 weeks) and have variable sensitivity (15-84%)⁴, the cytopathologic examination is faster but has an even lower sensitivity (9-50%), and the serum and urine antigens have better sensitivity and specificity but there is cross-reactivity with other fungi and they can take days to process as they are not routine tests.

The present inventors have developed a breath based, noninvasive, point-of-care diagnostic test for pulmonary and disseminated histoplasmosis via the identification of volatile organic compounds (VOCs).

As described herein, the present inventors have identified a unique and specific VOC profile of Histoplasma capsulatum that includes several volatile sesquiterpene compounds that can be used to discriminate this species from other fungal species, and demonstrated that GC-MS can be used for the rapid discrimination of fungal species using pattern-based identification of these species-specific VOC profiles. The sesquiterpene compounds were present in the breath of patients with histopasmosis. A combination of Cyperene; (1R,4aR,8aR)-2,5,5,8a Tetramethyl-4, 5,6,7,8,8a-hexahydro-1H-1,4a-methanonaphthalene; and viridiflorol accurately distinguished patients with histoplasmosis from patients with other fungal infections, TB, and pneumonia, with 100% sensitivity and 81% specificity, respectively.

Detection of these unique VOC profiles can be harnessed for direct detection of these fungal volatile profiles in the breath of patients with pulmonary or disseminated histoplasmosis and can be used for the rapid and noninvasive diagnosis of pulmonary or disseminated histoplasmosis. The methods and devices described herein, e.g., the GC-MS or DMS-based detection methods, can be adapted to a small, portable bedside breath gas detection system for real-time patient breath surveillance for this pattern of fungal metabolites, to allow for earlier histoplasmosis diagnosis than currently possible, more rational test-based prescribing of antifungal medications, monitoring of clinical response to antifungal therapy, and ultimately, better patient outcomes.

As described herein, among other uses, these VOC profiles can be used for:
a. rapid, noninvasive, and sensitive breath tests for the diagnosis of pulmonary or disseminated histoplasmosis and the discrimination of histoplasmosis from other fungal infections;
b. surrogate marker demonstrating successful antifungal treatment of histoplasmosis, and
c. rapid identification and antifungal susceptibility testing of Histoplasma capsulatum, e.g., in the microbiology laboratory, based on their VOC profile (i.e., the VOCs present in the sample).

### Histoplasmosis

The methods described herein can be used to detect or pulmonary or disseminated histoplasmosis in a subject, to select treatment and to treat pulmonary or disseminated histoplasmosis, and to monitor treatment of pulmonary or disseminated histoplasmosis.

Histoplasmosis is a fungal infection caused by Histoplasma capsulatum. Symptoms of this infection vary greatly, but the disease affects primarily the lungs, resulting in pulmonary histoplasmosis. Occasionally, other organs are affected; called disseminated histoplasmosis, it can be fatal if left untreated.

### Samples

The methods described herein can be performed on a gas or liquid sample. In some embodiments, the sample is exhaled breath directly from an individual or from a breathing machine such as a ventilator. Alternatively, the methods can be performed using headspace from a culture known or suspected to include Histoplasma capsulatum, e.g., commercially-available or lab-cultured species or species obtained from a primary sample from a subject, e.g., a clinical sample obtained by biopsy of the affected area (e.g., nasal biopsy, transthoracic percutaneous needle aspiration, or video assisted thoracoscopic biopsy) or bronchoalveolar lavage. The sample is maintained in a suitable growth medium to allow growth and metabolism of any Histoplasma capsulatum in the sample. In certain embodiments, the invention involves taking a clinical sample from a subject and placing it in media, for example, with microfluidics, or in culture, for example, with conventional culturing methods. The Histoplasma capsulatum, if present, are stimulated to metabolize. The headspace (gaseous phase) generated as a result of this metabolism can be collected and analyzed using a method described herein or known in the art, see, e.g., US20100291617. In some embodiments, the methods are performed directly on bronchoalveolar washings, obtained by bronchoscopy/bronchoalveolar lavage. In some embodiments, the sample is a gas, e.g., gas from the headspace of an in vitro culture sample. Where headspace gas is used, the gas should be collected after the headspace has been in contact with the culture for a sufficient amount of time for the compounds to be present, preferably in an air-tight, sealed environment. In some embodiments, the gas is patient breath (e.g., tidal breath from spontaneously breathing patients).

The VOCs can also be detected in a liquid sample, since they are expected to be there in equilibrium with the gaseous phase. Thus, in addition to or as an alternative, the samples assayed using the methods described herein can include a liquid, e.g., blood (e.g., plasma or serum), lymph, urine, tears, saliva, sputum, nasal mucus, phlegm (e.g., expectorate), or CSF from a subject (e.g., from a biological fluid that comes near or preferably into contact with the tissue or organ that is known or suspected to be infected with Histoplasma capsulatum), or the liquid phase (e.g., supernatant) of an in vitro culture. In some embodiments, the sample comprises saliva from the subject.

### Detection Methods

A number of methods known in the art can be used to detect the presence of the VOCs described herein in a sample. Exemplary methods (particularly for use with a gas sample) include gas chromatography (GC); spectrometry, for example mass spectrometry (including quadrapole, time of flight, tandem mass spectrometry, ion cyclotron resonance, and/or sector (magnetic and/or electrostatic)), ion mobility spectrometry, field asymmetric ion mobility spectrometry, and/or DMS; fuel cell electrodes; light absorption spectroscopy; nanoparticle technology; flexural plate wave (FPW) sensors; electrochemical sensors; photoacoustic equipment; laser-based equipment; electronic noses (bio-derived, surface coated); and various ionization techniques. See, e.g., US20100291617 and US20070003996. In some embodiments, the method is GC-MS. Preferred methods include ion mobility spectrometry (IMS) or differential mobility spectrometry (DMS).

In some embodiments, the methods described herein include the use of differential mobility spectrometry to detect VOCs in a sample. An exemplary micro-machined differential mobility spectrometer (DMS), developed for chemical and biological sensing applications, is currently available from Sionex Corporation. DMS has several features that make it an excellent platform for VOC analysis: it is quantitative, selective, and exquisitely sensitive, with a volatile detection limit in the parts-per-trillion range (Davis et al., In: 12th International Conference on Transducers, Solid-State Sensors, Actuators and Microsystems; 2003; p. 1233-8 vol.2; Miller et al., In: Solid-State Sensors and Actuators Workshop; 2000; Hilton Head, South Carolina; 2000; Krebs et al., Sensors Journal, IEEE 2005; 5(4):696-703). Unlike mass spectrometry, which separates particles based on mass/charge ratios, DMS harnesses differences in ion mobility in low and high electric fields to achieve a gas-phase separation of ions at atmospheric pressure. DMS rapidly detects compounds that are difficult to resolve by other analytical techniques such as mass spectrometry in challenging matrices such as human breath (Kanu et al., J Mass Spectrom 2008; 43:1-22; Kanu et al., J Chromatogr A 2008; 1177:12-27; Luong J et al., J Chromatogr Sci 2006; 44:276-286; Nazarov et al., Anal Chem 2006; 7697-706; Kolakowski et al., Analyst 2007; 132:842-64).

DMS can be tuned to monitor specific ion masses, thus tailoring response characteristics to focus on various compounds of interest. It requires no reagents, generates the high fields required by the sensor using a small power supply, and has already been microfabricated, resulting in a small, portable machine that can be used at the bedside, with a turnaround time of several minutes. DMS has been used successfully in several commercial settings, including a hand-held, portable detector of trace levels of chemical warfare agents from General Dynamics (JUNO™) and airport explosives detectors from Thermo (see, e.g., U.S. Pat. No. 7,605,367). DMS technology has also been successfully applied to the characterization of unique VOCs produced by Mycobacterium tuberculosis and other bacteria (Fong et al., Anal Chem 2011; 83:1537-46; Shnayderman et al., Anal Chem 2005; 77:5930-7).

To perform a measurement using a DMS, a gas sample is introduced into the spectrometer, where it is ionized, and the ions are transported through an ion filter towards the detecting electrodes (Faraday plates) by a carrier gas. The DMS device can separate chemical components of a substance based on differing ion mobilities. For other devices, measurements are performed using methods known in the art.

Additional non-limiting examples of systems that can be used in the present methods include those described in US20090078865; US20130168548; US20100291617 and US20070003996.

In some embodiments, the methods include obtaining a sample of ambient air and detecting the presence and/or levels of VOCs in the air, to provide a reference for subtraction of ambient VOCs.

A number of methods are known in the art for detecting the presence and/or levels of the VOCs in a liquid sample, including but not limited to chromatography (e.g., HPLC) and spectrophotometry (e.g., MS, LC-MS, MALDI-TOF, and other of the methods described above for gas-phase samples).

### Combination Diagnostics

In some embodiments, the methods include performing an additional diagnostic test for Histoplasma capsulatum. A number of such tests are known in the art and include galactomannan enzyme immunoassays; radiology imaging studies (e.g., CT imaging); bronchoalveolar lavage, transthoracic percutaneous needle aspiration, or video assisted thoracoscopic biopsy; urinary or serum antigen tests. A positive result on one of these tests can provide further evidence supporting a diagnosis of Histoplasma capsulatum; see, e.g., www.cdc.gov/fungal/diseases/histoplasmosis/diagnosis.html.

### Histoplasma Capsulatum Identification and Diagnosis

As described herein, Histoplasma capsulatum produces VOCs that can be used to identify it in a sample, e.g., in a sample comprising breath of a subject, or headspace from a culture suspected of comprising Histoplasma capsulatum; the culture can be, e.g., a culture of a biopsy from a subject, or a culture in a microbiology laboratory, e.g., a culture known or suspected of containing or being contaminated with Histoplasma capsulatum. This identification can be used to diagnose a subject with Histoplasma capsulatum, allowing for the quick and efficient administration of treatments, e.g., as described below.

Thus, the methods described herein can include obtaining a sample comprising breath of a subject, or headspace from a culture suspected of comprising Histoplasma capsulatum, and detecting and identifying the VOCs in the sample. For example, the methods can include detecting the presence of one, two, or all three of cyperene, 1R,4aR,8aR)-2,5,5,8a-Tetramethyl-4,5,6,7,8,8a-hexahydro-1H-1,4a-methanonaphthalene, and viridiflorol in the sample.

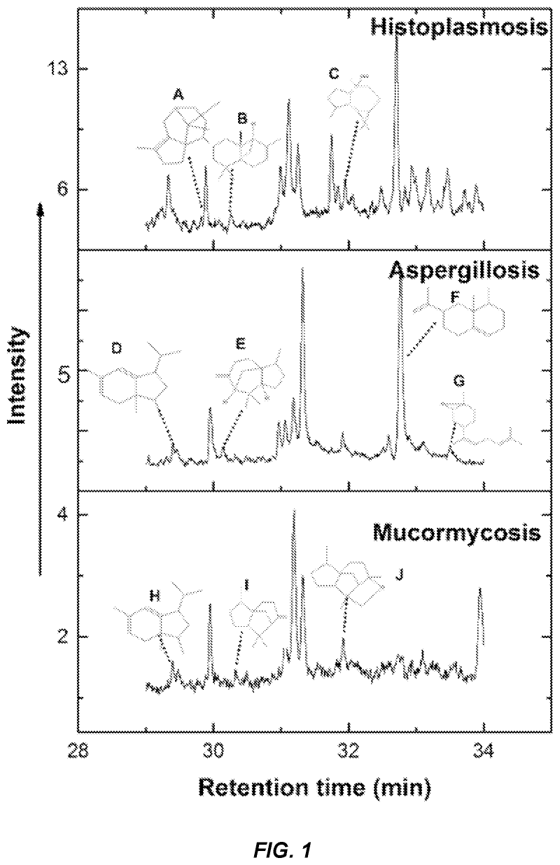

In some embodiments, the methods described herein can be used to distinguish or differentiate patients having either histoplasmosis or another fungal infection (e.g., tuberculosis, aspergillosis, mucormycosis, molds (Scedosporium, Fusarium, Penicillium, Scopulariopsis, Syncephalastrum), endemic fungi (Paracoccidioides brasiliensis), bacterial pneumonia (Staphylococcus aureus, Klebsiella pneumoniae, Pseudomonas aeruginosa, Stenotrophomonas maltophilia, Acinetobacter baumannii, Haemophilus influenzae, Escherichia coli, Streptococcus pneumoniae, Burkholderia cepacia). Specifically, the present inventors have discovered that a unique VOC profile of Histoplasma capsulatum. The unique profile may allow one of skill in the art to quickly identify a subject infected with Histoplasma capsulatum based on which VOCs are identified in the subject's breath (e.g. the presence of one, two, or all three of cyperene, 1R,4aR,8aR)-2,5,5,8a-Tetramethyl-4,5,6,7,8,8a-hexahydro-1H-1,4a-methanonaphthalene, and viridiflorol). For example, in some embodiments, diagnosis of a subject with histoplasmosis requires that the sample obtained from the subject has VOC metabolites as shown by peaks A, B, and C in FIG. 1 (e.g., and wherein the sample does not have the VOC metabolites as shown by peaks D, E, F, G, H, or I).

### Methods of Treatment

The methods described herein can be used to select a treatment for a subject, and can optionally include administering the treatment to a subject. When a subject has been diagnosed by a method described herein as having histoplasmosis, then a treatment comprising administration of a therapeutically effective amount of an antifungal compound can be administered.

A number of antifungal compounds are known in the art and under development. At present, deoxycholate amphotericin B (D-AMB) and its lipid formulations (AMB lipid complex (ABLC), liposomal amphotericin B (LAMB), and Amphotericin B cholesteryl sulfate complex (AMB colloidal dispersion, ABCD)); azole compounds (itraconazole, voriconazole, posaconazole); and echinocandins (caspofungin, micafungin, anidulafungin) are in clinical use.

In some embodiments, the methods include selecting and optionally administering an azole antifungal, e.g., itraconazole (ITR), voriconazole (VOR), posaconazole (POS), ravuconazole (RAV), or isavuconazole (ISA), or an amphotericin B (AMB) formulation as described above, to a subject identified by a method described herein as having histoplasmosis. In some embodiments, the methods include administering an echinocandin, e.g., caspofungin, micafungin or anidulafungin, e.g., alone or in combination with an azole (e.g., voriconazole) or AMB.

In some embodiments, the methods described herein can be used to determine susceptibility of Histoplasma capsulatum, e.g., to treatment with a known or suspected antifungal, e.g., in the microbiology laboratory. A sample suspected or known to include Histoplasma capsulatum from a subject is obtained and cultured as described above, e.g., under conditions mimicking the in vivo environment, and then exposed to a potential treatment (e.g., a known or experimental treatment). After exposure to the treatment, the VOCs present in the headspace of the culture are sampled. If the treatment decreases VOCs as compared to a reference level (e.g., a level of VOCs in the headspace before exposure to the treatment), then the Histoplasma capsulatum in the sample is considered susceptible to the treatment. In this case, the treatment is likely to be effective in treating histoplasmosis in the subject; the treatment can be selected and optionally administered to subject.

### Monitoring Treatment Efficacy

As described herein, successful treatment of a Histoplasma capsulatum infection results in a decrease in fungal VOCs. Thus, the methods can include repeated assays of VOC levels in a subject, e.g., before, during, and after administration of a treatment for histoplasmosis. A decrease in VOC levels would indicate that the treatment has been successful. In some embodiments, levels of one, two, or all three of cyperene, 1R,4aR,8aR)-2,5,5,8a-Tetramethyl-4,5,6,7,8,8a-hexahydro-1H-1, 4a-methanonaphthalene, and viridiflorol are determined.

### Methods of Identifying Novel Antifungal Agents

Included herein are methods for screening test compounds, e.g., polypeptides, polynucleotides, inorganic or organic large or small molecule test compounds, to identify agents useful in the treatment of histoplasmosis.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample comprising Histoplasma capsulatum, and the ability of the test compound to decrease levels of a VOC as described herein in the headspace of the culture is determined.

In some embodiments, the test sample is, or is derived from (e.g., a sample taken from) an in vivo model of a disorder as described herein. For example, an animal model, e.g., a rodent (such as a rat or mouse) that has been infected with Histoplasma capsulatum can be used.

A test compound that has been screened by a method described herein and determined to decrease VOCs, can be considered a candidate compound. A candidate compound that has been screened, e.g., in an in vivo model of a disorder, e.g., a rodent infected with Histoplasma capsulatum, and determined to decrease VOCs in a sample comprising breath from the infected animal model or headspace from a culture of a sample from the infected animal model, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that decrease fungal VOCs in an animal model) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating histoplasmosis. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of histoplasmosis, as described herein. The animal can be monitored for a change in the disorder, e.g., for an improvement in a parameter of the disorder, e.g., a parameter related to clinical outcome. In some embodiments, the parameter is VOCs or survival, and an improvement would be a reduction in VOCs or an increase in survival. In some embodiments, the subject is a human, e.g., a human with histoplasmosis and the parameter is levels of fungal VOCs or survival.

### EXAMPLES

The invention is further described in the following examples, which do not limit the scope of the invention described in the claims.

### Example 1. Breath Signature for the Diagnosis of Pulmonary and Disseminated Histoplasmosis

Looking for a faster and easier way to identify histoplasmosis, we developed a breath based, noninvasive, point-of-care diagnostic test for pulmonary and disseminated histoplasmosis via the identification of volatile organic compounds (VOCs) hoping to characterize a specific breath profile for this fungus to ease diagnosis in the hospital setting but also in underserved areas where there is a high disease burden.

### Methods

### Study Subjects

We enrolled 143 patients across two health centers: 56 with suspected histoplasmosis from March 2019 to February 2020 at Hospital Roosevelt (HR; Guatemala City, Guatemala), and 87 with suspected invasive fungal disease from July 2016 to October 2019 at Brigham and Women's Hospital (BWH; Boston, MA, USA).

Inclusion Criteria: Patients 18 years old or older who could provide informed or surrogate consent, with suspected histoplasmosis based on epidemiology, risk factors, clinical, and imaging findings.

Exclusion Criteria: Patients without respiratory symptoms, and/or unable to provide informed or surrogate consent.

We obtained written informed consent from participating subjects with the Spanish equivalent in the Guatemalan cohort, and the protocol was approved by the institutional review board at both centers.

Table 1 below is a summary of the cohort's characteristics and Table 2 below lists the number of patients in the cohort that had a diagnosis of histoplasmosis or other infections.

TABLE 1
Cohort characteristics

| | N | % |
|---|---|---|
| Median age (years), IQR | 52 | 28.5 |
| Male | 107 | 74.8 |
| HIV/AIDS | 24 | 16.8 |
| Hematologic malignancy | 62 | 43.4 |
| Hematologic cell transplantation | 21 | 14.7 |
| Solid organ transplantation | 12 | 8.4 |
| Other immunosuppressant | 2 | 1.4 |
| exposure* | | |

*Exposure to ≥20 mg of prednisone or equivalent for >2 weeks, T-cell immunosuppressants.

TABLE 2
Diagnosis of histoplasmosis or other infections

| | N |
|---|---|
| Proven or probable histoplasmosis\hat{ } | 8 |
| Tuberculosis | 13 |
| Proven or probable invasive aspergillosis | 22 |
| Mucormycosis | 3 |
| Other molds^{¥} | 5 |
| Other endemic fungi* | 1 |
| Bacterial pneumonia** | 13 |
| No microbiology identified | 50 |

\hat{ }3 disseminated, 5 pulmonary; 4 patients with histoplasmosis had co-infections (2 tuberculosis, 1 influenza, 1 Pneumocystis jirovecii pneumonia; 4 patients were receiving empiric antifungal therapy active against Histoplasma at the time of their first breath sample collection.
^{¥}Scedosporium, Fusarium, Penicillium, Scopulariopsis, Syncephalastrum.
*Paracoccidioides brasiliensis.
**Staphylococcus aureus, Klebsiella pneumoniae, Pseudomonas aeruginosa, Stenotrophomonas maltophilia, Acinetobacter baumannii, Haemophilus influenzae, Escherichia coli, Streptococcus pneumoniae, Burkholderia cepacia.

Sample collection and processing

We collected 4-minutes of tidal breath from spontaneously breathing patients with adsorption of volatile organic compounds (VOCs) into two parallel thermal desorption tubes (containing tandem beds of Tenax TA, Carbograph 1 TD, and Carboxen 1003) using an air sampling pump calibrated to 900 mL per minute. Samples were collected at baseline, and then 3, 7, 14, and 28 days after starting antifungal therapy when possible. The compounds were thermally desorbed onto an automated thermal desorption unit and gas chromatography (GC) unit interfaced to a triple quadrupole mass spectrometry (MS) detector. One investigator performed the identification of GC-MS peaks using the National Institute of Standards and Technology (NIST) 17 Mass Spectral Library and MassHunter Software (Ver. 7.0.0. Agilent Technologies, Inc.).

### Patient Classification

The patients were classified as having proven (positive histopathology (intracellular yeast forms), microscopy or culture from the affected site or blood), probable (environmental exposure to the fungus and compatible clinical illness, positive urine, serum or body fluid Histoplasma antigen), or no histoplasmosis following the EORTC/MSG consensus definitions for endemic fungal infections.⁵ For the purposes of our study, both proven or probable were considered to have histoplasmosis, and other patients with other invasive mycoses, bacterial infections or tuberculosis, were considered not to have histoplasmosis.

### Results

The median age was 52 years, 107 (74.8%) were male, and 36 (25.2%) were female. 24 (16.8%) had HIV, 62 (43.4%) had hematologic malignancies, and 21 (14.7%) were stem cell transplant recipients.

Eight patients were diagnosed with histoplasmosis over the study period (four at HR, four at BWH), with a clinical syndrome and a positive Histoplasma urine or serum antigen test. One patient also had yeast forms on tissue biopsy. 3 patients had disseminated and 5 pulmonary histoplasmosis. 4 patients with histoplasmosis had co-infections—2 tuberculosis (TB), 1 influenza, and 1 Pneumocystis jirovecii (PJP) pneumonia. 4 patients were receiving antifungal therapy active against Histoplasma at the time of their first breath sample.

We found 3 sesquiterpenes: (A) cyperene, (B) 1R,4aR,8aR)-2,5,5,8a-Tetramethyl-4, 5,6,7,8,8a-hexahydro-1H-1,4a-methanonaphthalene, and (C) viridiflorol in patients with histoplasmosis, that distinguished these patients from those with other pneumonia (TB, paracoccidioidomycosis, coccidioidomycosis, invasive aspergillosis, mucormycosis, PJP, bacterial pneumonia) with variable sensitivity and specificity depending on the compounds selected for the calculations. The sensitivity of any of these three sesquiterpene compounds in the breath was 100% (95% CI 59-100) for histoplasmosis, with a specificity of 95% (95% CI 89-97).

FIG. 1 shows the GC-MS peaks for the compounds and their molecular structures as well as the differences with compounds present in the breath of patients with invasive aspergillosis and mucormycosis.

### Discussion

Our study shows that there is a unique secondary metabolite breath signature that can be used for the noninvasive diagnosis of pulmonary and disseminated histoplasmosis. The fact that the breath metabolomic profile is clearly distinct from pulmonary tuberculosis and mycoses such as aspergillosis, mucormycosis, and paracoccidioidomycosis is very promising and we aim in the future to translate the GC-MS findings to a DMS (differential mobility spectrometry) portable microanalyzer to use as a point-of-care tool for fast and accurate diagnosis to guide therapy initiation and improve outcomes.

### REFERENCES

1. Scully M C, Baddley J W. Epidemiology of Histoplasmosis. Curr Fungal Infect Rep. 2018;12(1):51-58. doi:10.1007/s12281-018-0309-x.
2. Azar M M, Loyd J L, Relich R F, Wheat L J, Hage C A. Current Concepts in the Epidemiology, Diagnosis, and Management of Histoplasmosis Syndromes. Semin Respir Crit Care Med. 2020;41(1):13-30. doi:10.1055/s-0039-1698429.
3. Baddley J W, Winthrop K L, Patkar N M, et al. Geographic Distribution of Endemic Fungal Infections among Older Persons, United States. Emerg Infect Dis J. 2011;17(9):1664. doi:10.3201/eid1709.101987.
4. Poplin V, Smith C, Milsap D, Zabel L, Bahr N C. Diagnosis of Pulmonary Infections Due to Endemic Fungi. Diagnostics (Basel, Switzerland). 2021;11(5):856. doi:10.3390/diagnostics11050856.
5. Donnelly J P, Chen S C, Kauffman C A, et al. Revision and Update of the Consensus Definitions of Invasive Fungal Disease From the European Organization for Research and Treatment of Cancer and the Mycoses Study Group Education and Research Consortium. Clin Infect Dis. 2020;71(6):1367-1376. doi:10.1093/cid/ciz1008.
6. Connolly P A, Durkin M M, Lemonte A M, Hackett E J, Wheat L J. Detection of histoplasma antigen by a quantitative enzyme immunoassay. Clin Vaccine Immunol. 2007;14(12):1587-1591. doi:10.1128/CVI.00071-07.
7. Cáceres D H, Gómez B L, Tobón A M, Chiller T M, Lindsley M D. Evaluation of a Histoplasma antigen lateral flow assay for the rapid diagnosis of progressive disseminated histoplasmosis in Colombian patients with AIDS. Mycoses. 2020;63(2):139-144. doi:10.1111/myc.13023.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for diagnosing a subject with histoplasmosis, the method comprising: obtaining a sample comprising breath of a subject or suspected of comprising Histoplasma capsulatum isolated from a subject;
detecting the presence in the sample of one, two, or all three volatile organic compounds (VOCs) produced by Histoplasma capsulatum in a sample comprising breath from the subject or headspace from a culture suspected of comprising Histoplasma capsulatum isolated from the subject, wherein the VOCs are selected from the group consisting of cyperene, 1R,4aR,8aR)-2,5,5,8a-Tetramethyl-4, 5,6,7,8,8a-hexahydro-1H-1,4a-methanonaphthalene, and viridiflorol; and
diagnosing a subject as having histoplasmosis when there are one, two, or all three of the VOCs present in the sample.

2. A method of treating a subject who has histoplasmosis, the method comprising: obtaining a sample comprising breath of a subject or headspace from a culture suspected of comprising Histoplasma capsulatum isolated from a subject;
detecting the presence in the sample one, two, or all three VOCs selected from the group consisting of cyperene, 1R,4aR,8aR)-2,5,5,8a-Tetramethyl-4,5,6,7,8,8a-hexahydro-1H-1, 4a-methanonaphthalene, and viridiflorol; and
administering an antifungal treatment to a subject who has one, two, or all three VOCs selected from the group consisting of cyperene, 1R,4aR,8aR)-2,5,5,8a-Tetramethyl-4, 5,6,7,8,8a-hexahydro-1H-1,4a-methanonaphthalene, and viridiflorol.

3. The method of any one of claim 1 or 2, wherein the sample is a sample of a subject's breath.

4. A method of monitoring efficacy of a treatment for histoplasmosis in a subject, the method comprising:
determining a first level of one, two, or all three volatile organic compounds (VOCs) produced by Histoplasma capsulatum in a sample comprising breath from the subject or headspace from a culture suspected of comprising Histoplasma capsulatum isolated from the subject, wherein the VOCs are selected from the group consisting of cyperene, 1R,4aR,8aR)-2,5,5,8a-Tetramethyl-4,5,6,7,8,8a-hexahydro-1H-1, 4a-methanonaphthalene, and viridiflorol, in the subject;
administering a treatment for histoplasmosis to the subject;
determining a second level of the VOCs in a sample obtained after administration of the treatment to the subject; and
comparing the first and second levels of VOCs, wherein a decrease in the VOCs indicates that the treatment has been effective in treating the histoplasmosis in the subject, and an increase or no change indicates that the treatment has not been effective in treating the histoplasmosis in the subject.

5. The method any one of claims 2 to 4, wherein the treatment comprises administration of one or more doses of one or more antifungal compounds.

6. A method of identifying a candidate compound for the treatment of histoplasmosis, the method comprising:
providing a test culture comprising Histoplasma capsulatum;
detecting a baseline level of fungal VOCs in the headspace of the culture in the absence of the test compound, wherein the VOCs are selected from the group consisting of cyperene, 1R,4aR,8aR)-2,5,5,8a-Tetramethyl-4,5,6,7,8,8a-hexahydro-1H-1, 4a-methanonaphthalene, and viridiflorol, in the subject;
contacting the test culture with a test compound;
determining a second level of the VOCs in a the test culture;
comparing the second level of VOCs to the baseline level; and
identifying a test compound that decreases levels of fungal VOCs in the test culture as a candidate compound for the treatment of histoplasmosis.

7. The method of any of claims 1-6, wherein determining the presence of a VOC comprises assaying the sample to detect the presence the VOC.

8. The method of claim 7, wherein assaying the sample to detect the presence the VOC comprises using a gas chromatography-mass spectrometry (GC-MS) method.

9. The method of claim 8, wherein the spectrophotometry method is mobility spectrometry (IMS) or differential mobility spectrometry (DMS).

10. The method of any of claim 1-5 or 7-9, wherein the subject is a human.

11. The method of claim 10, wherein the subject is a human.

12. The method of claim 5, wherein the antifungal compound is amphotericin B.

13. The method of claim 5, wherein the antifungal compound is an azole antifungal compound.

14. The method of claim 13 wherein the azole compound is itraconazole.

15. The method of claim 5, wherein the antifungal compounds are amphotericin B and an azole antifungal compound.
